(19) United States
(12) Patent Application Publication
(10) Pub. No.: US 2025/0135223 A1
(43) Pub. Date: May 1, 2025

(54) Neurological Brain Entrainment Device

(71) Applicant: Ali-Rashad Richey, McDonough, GA (US)

(72) Inventor: Ali-Rashad Richey, McDonough, GA (US)

(21) Appl. No.: 18/929,170

(22) Filed: Oct. 28, 2024

Related U.S. Application Data
(60) Provisional application No. 63/593,427, filed on Oct. 26, 2023.

(51) Int. Cl. A61N 5/06; A61M 21/00

(52) U.S. Cl. CPC A61N 5/0622; A61M 21/00; A61M 2021/0022; A61M 2021/0027; A61M 2205/50; A61M 2205/8206; A61M 2205/8256; A61M 2209/088; A61M 2210/0618; A61M 2210/0662; A61M 2210/12; A61N 2005/0607; A61N 2005/0626; A61N 2005/0632; A61N 2005/0648; A61N 2005/0663

(57) ABSTRACT

A neurological brain entrainment device is an apparatus used to synchronize a user's brainwaves through external stimuli. The apparatus includes a headband, a left earphone, a right earphone, at least one vibrational module, a controller, at least one user control, and a power source. The headband secures the apparatus around a user's head. The left earphone and the right earphone are used to provide auditory stimuli to a user's ears. The vibrational module is used to provide tactile stimuli to a user's temples. The controller is used to manage the other electronic components of the apparatus. The user control allows a user to enter their input into the apparatus. The power source is used to provide electrical power to the other electronic components of the apparatus.

The current application claims a priority to the U.S. provisional patent application Ser. No. 63/593,427 filed on Oct. 26, 2023. The current application is filed on Oct. 28, 2024, while Oct. 26, 2024 was on a weekend.

### FIELD OF THE INVENTION

The present invention generally relates to brainwave entrainment. More specifically, the present invention is a device that effects frequencies through sound, vibration, and light for brainwave entrainment.

### BACKGROUND OF THE INVENTION

Brainwave entrainment, also referred to as brainwave synchronization or neural entrainment, refers to the observation that brainwaves (i.e., large-scale electrical oscillations in the brain) will naturally synchronize to the rhythm of periodic external stimuli, such as flickering lights, speech, music, or tactile stimuli. As different conscious states can be associated with different dominant brainwave frequencies, it is hypothesized that brainwave entrainment might induce a desired state.

In order provide these stimuli in a convenient manner, the present invention provides a neurological brain entrainment device, which may comprise a headband, an adjustment module, an earphone, a vibrational band, a photo biomodulator, an intranasal device, a plurality of buttons, and a charging port. The headband is placed on the head of a user so that the headband is positioned over the eyes and forehead and is wrapped around the head. Two bio-formed earphones are positioned within the headband on either side of the user's head, over the ears. The earphones deliver sound frequency. Two vibrational bands may be positioned within the headband, one on either side of the user's head. The vibrational bands may correlate their vibrational frequency to the vibrational frequency of the earphones. The photo biomodulator may emit photonic light towards the user.

In some embodiments of the present invention, the internasal device may be a detachable or separate component from the headband. The intranasal device is positioned within the nose of the user and emits the same light in the same frequency as the photo biomodulator. The brain entrainment device may be powered by a battery, and the battery is rechargeable via a charging cable. Buttons may be positioned on the brain entrainment device to allow the user to adjust or select various settings.

### BRIEF DESCRIPTION OF THE DRAWINGS

FIG. 1 is a top perspective view of the present invention.

FIG. 2 is bottom perspective view of the present invention.

FIG. 3 is a left view of the present invention.

FIG. 4 is a right view of the present invention.

FIG. 5 is an exploded top perspective view of the present invention.

FIG. 6 is a top schematic view of the present invention.

FIG. 7 is a schematic diagram of the electronic connections between components of the present invention.

FIG. 8 is a schematic diagram of the electrical connections between components of the present invention.

### DETAILED DESCRIPTION OF THE INVENTION

All illustrations of the drawings are for the purpose of describing selected versions of the present invention and are not intended to limit the scope of the present invention.

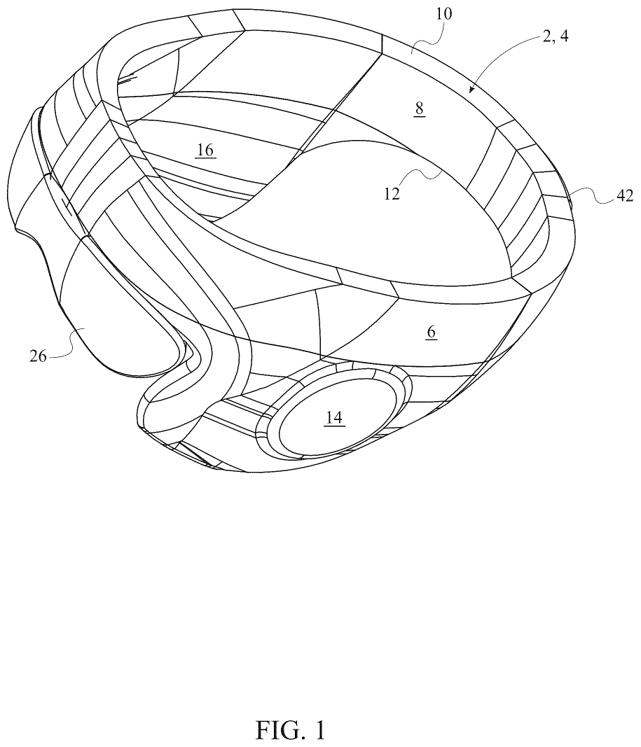

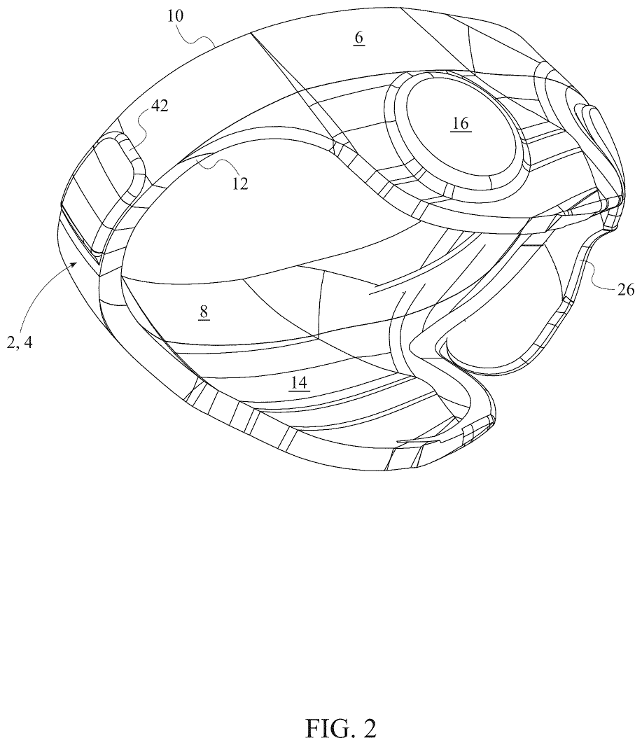

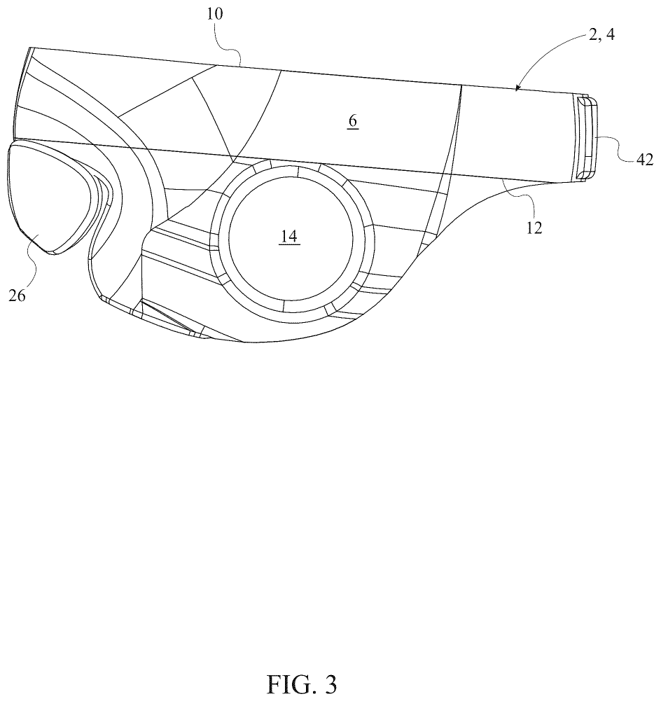

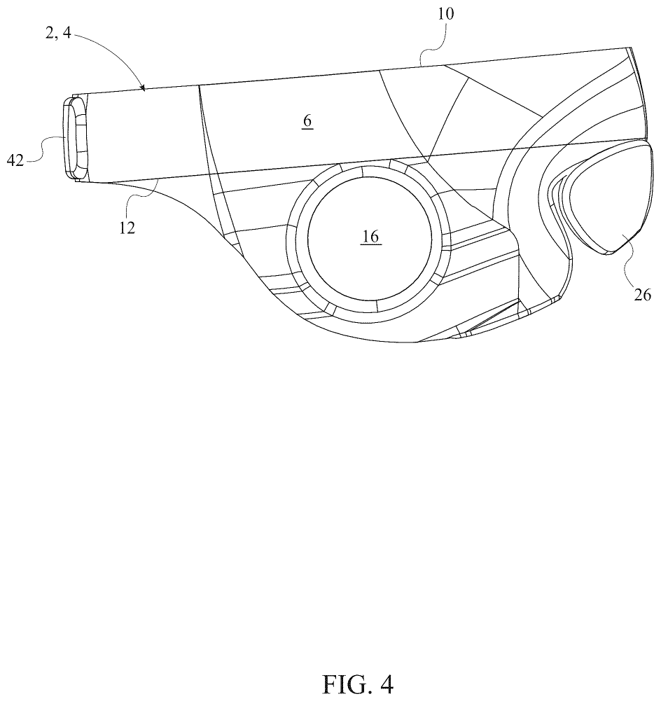

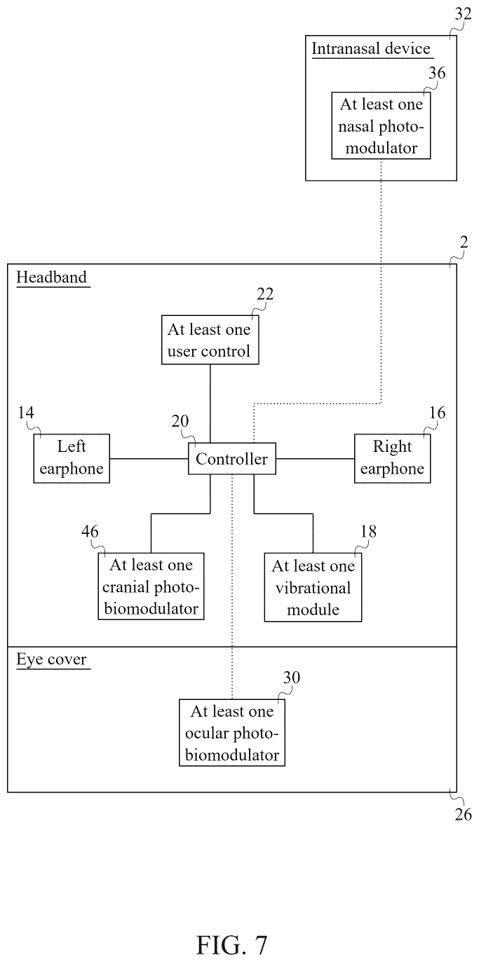

The present invention is a neurological brain entrainment device that is used to synchronize a user's brainwaves through external stimuli. Thus, the present invention comprises a headband 2, a left earphone 14, a right earphone 16, at least one vibrational module 18, a controller 20, at least one user control 22, and a power source 24, which are shown in FIGS. 1 through 8. The headband 2 is used to restrain the present invention around a user's head. The headband 2 is preferably made of any elastic fabric. The left earphone 14 is used to provide auditory stimuli to a user's left ear, while the right earphone 16 is used to provide auditory stimuli to a user's right ear. The at least one vibrational module 18 is used to provide tactile stimuli about a user's head. The controller 20 is used to manage the other electronic components of the present invention. The controller 20 is preferably a chipset (e.g., a motherboard) with a microprocessor and a wireless communication module [e.g., a personal area network (PAN) communication module, a local area network (LAN) communication module, etc.]. The at least one user control 22 allows the present invention to receive a user input. The at least one user control 22 is preferably a set of buttons, each of which relates to a specific functionality of the present invention (e.g., turning on/off the present invention, specifically activating/deactivating earphones, specifically activating/deactivating photo-biomodulators, specifically activating/deactivating a vibrational module, setting a time period for a therapeutic session by the present invention, initiating a sleep mode for the present invention, etc.). The power source 24 is used to electrically power all of the electronic components of the present invention.

The headband 2 is also used as a base to connect the other components of the present invention together. Thus, the headband 2 comprises some reference components including a band body 4, an outer band surface 6, an inner band surface 8, an upper band rim 10, and a lower band rim 12. The band body 4 is the annular structural body of the headband 2. The inner band surface 8 is a smaller annular surface that is encircled by the outer band surface 6, which is a larger annular surface. The upper band rim 10 and the lower band rim 12 are two circular edges that are positioned opposite to each other across a height of the headband 2, and these two circular edges preferably have the same circumference and preferably are concentric to each other.

The general configuration of the aforementioned components allows the present invention to efficiently and effectively synchronize a user's brainwaves through external stimuli. The left earphone 14 and the right earphone 16 are integrated through the lower band rim 12 and into the band body 4 and are positioned opposite to each other about the band body 4, which allows the present invention to anatomically place the left earphone 14 over a user's left ear and to anatomically place the right earphone 16 over the user's right ear as the headband 2 is worn about the user's head. The at least one vibrational module 18 is integrating about the band body 4 and is positioned adjacent to the left earphone 14 and the right earphone 16 in order to anatomically place the at least one vibrational module 18 near a user's temples. The at least one user control 22 is externally mounted to the outer band surface 6 so that a user is able to easily actuate the at least one user control 22. The controller 20 is housed within the band body 4 in order to protect the controller 20 from any external damage and in order to structurally hold the controller 20 in place. The power source 24 is integrated into the band body 4, which allows the power source 24 to be easily accessible by the other electronic components of the present invention and/or a user of the present invention. The controller 20 is electronically connected to the left earphone 14, the right earphone 16, the at least one vibrational module 18, and the at least one user control 22 so that the controller 20 is readily able to receive digital information and/or digital instructions from any of these electronic components and is readily able to send digital information and/or digital instructions to any of these electronic components. The power source 24 is electrically connected to the controller 20, the left earphone 14, the right earphone 16, the at least one vibrational module 18, and the at least one user control 22 so that the power source 24 is readily able to deliver electrical power to any of these electronic components.

In some embodiments of the present invention, the left earphone 14 and the right earphone 16 are further configured to better accommodate a user of the present invention. The left earphone 14 may be ergonomically shaped to securely fit into a user's left ear. The right earphone 16 may also be ergonomically shaped to securely fit into a user's right ear. Moreover, the left earphone 14 and the right earphone 16 are preferably configured to emit a 40-Hertz (Hz) sound.

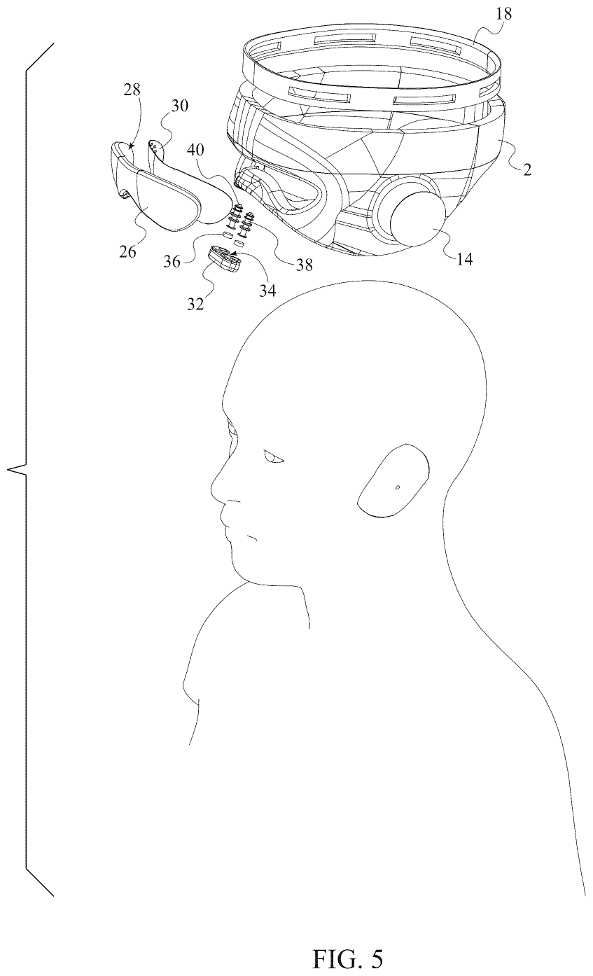

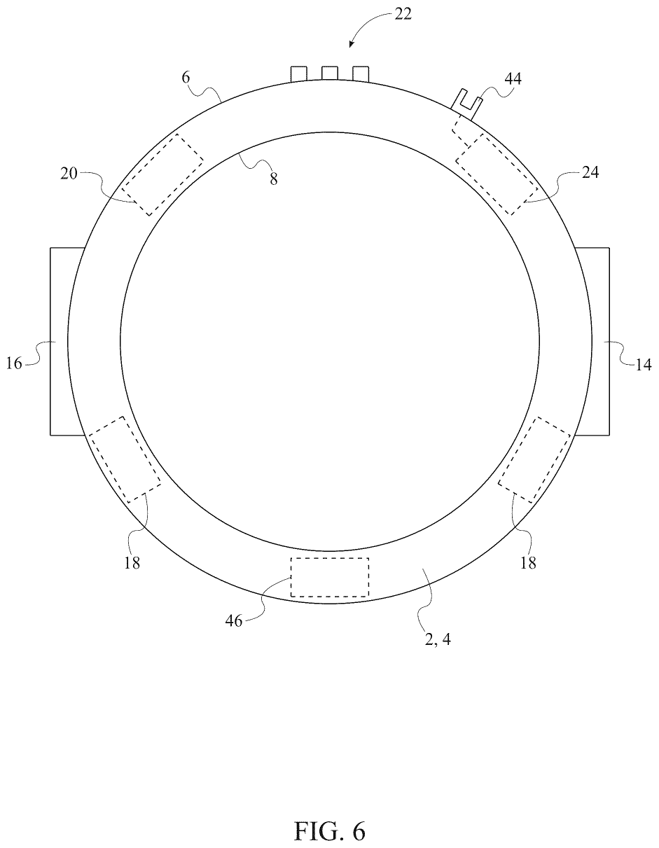

In some embodiments of the present invention, the at least one vibrational module 18 is further configured to better accommodate a user of the present invention. As can be seen in FIG. 5, the at least one vibrational module 18 may be a single continuous vibrational band that is integrated around the band body 4. As can be seen in FIG. 6, the at least one vibrational module 18 may alternatively be a left vibrational module and a right vibrational module. The left vibrational module and the right vibrational module are two discrete vibrational units that are positioned offset from each other about the band body 4. The left vibrational module would be used to provide tactile stimuli near a user's left temple, while the right vibrational module would be used to provide tactile stimuli near the user's right temple. Moreover, the at least one vibrational module 18 is preferably configured to vibrate at 40 Hz.

As can be seen in FIGS. 1 through 4, the present invention may further comprise an eye cover 26, which is used to prevent light from entering a user's eyes. The eye cover 26 is mounted adjacent to the lower band rim 12 and is positioned in between the left earphone 14 and the right earphone 16 about the band body 4, which allows the present invention to anatomically place the eye cover 26 over a user's eyes. The eye cover 26 can be a separate component that is not physically attached to the other components of the present invention. The eye cover 26 can alternatively be hingedly connected to the to the lower band rim 12 so that the eye cover 26 can readily be flipped up allowing a user to see with their eyes or can readily be flipped down allowing the user to prevent light from entering their eyes. Moreover, as can be seen in FIGS. 5, 7, and 8, the present invention may further comprise at least one ocular photo-biomodulator 30, which is used to stimulate a user's eyes in such a way to promote tissue repair, to reduce inflammation, to induce analgesia, to activate other therapeutic benefits, or a combination thereof. A proximal cover surface 28 of the eye cover 26 is preferably a concave surface located nearest to a user's eyes when the eye cover 26 is flipped down to cover the user's eyes. The at least one ocular photo-biomodulator 30 is mounted across the proximal cover surface 28 so that the at least one ocular photo-biomodulator 30 is able to directly emit therapeutic electromagnetic radiation into a user's eyes. The at least one ocular photo-biomodulator 30 can be communicably coupled to the controller 20 so that the controller 20 is able to manage and/or modify therapeutic electromagnetic radiation being emitted into a user's eyes by the at least one ocular photo-biomodulator 30. More specifically, if the eye cover 26 is not connected to the band body 4, the at least one ocular photo-biomodulator 30 can be communicably coupled to the controller 20 via a PAN communication module (e.g., Bluetooth). The at least one ocular photo-biomodulator 30 is preferably configured to emit electromagnetic radiation with a 660-nanometer (nm) wavelength.

As can be seen in FIGS. 5, 7, and 8, the present invention may further comprise an intranasal plug 32, which is used to prevent a smell from entering a user's nose. The intranasal plug 32 is positioned offset from the band body 4, which allows the present invention to anatomically place the intranasal plug 32 into a user's nose. The intranasal plug 32 can be a separate component that is not physically attached to the other components of the present invention. The intranasal plug 32 can alternatively be tethered to the band body 4 so that the intranasal plug 32 is not easily lost when the intranasal plug 32 is removed from a user's nose. Moreover, the present invention may further comprise at least one nasal photo-biomodulator 36, which is used to visually stimulate a user's nose in such a way to promote tissue repair, to reduce inflammation, to induce analgesia, to activate other therapeutic benefits, or a combination thereof. A proximal plug surface 34 of the intranasal plug 32 is preferably a surface located nearest to a user's nose when the intranasal plug 32 is inserted into the user's nose. The at least one nasal photo-biomodulator 36 is mounted across the proximal plug surface 34 so that the at least one nasal photo-biomodulator 36 is able to directly emit therapeutic electromagnetic radiation into a user's nose. The at least one nasal photo-biomodulator 36 can be communicably coupled to the controller 20 so that the controller 20 is able to manage and/or modify therapeutic electromagnetic radiation being emitted into a user's nose by the at least one nasal photo-biomodulator 36. More specifically, the at least one nasal photo-biomodulator 36 can be communicably coupled to the controller 20 via a PAN communication module (e.g., Bluetooth). The at least one nasal photo-biomodulator 36 is preferably configured to emit electromagnetic radiation with a 660-nm wavelength.

As can be seen in FIG. 5, the present invention may further comprise a left disposable nostril insert 38 and a right disposable nostril insert 40, each of which is a protrusion stuck into a respective nostril in order to secure the intranasal plug 32 to a user's nose. The left disposable nostril insert 38 and the right disposable nostril insert 40 are attached onto the proximal plug surface 34, which allows the present invention to anatomically place the left disposable nostril insert 38 into a user's left nostril and to anatomically place the right disposable nostril insert 40 into the user's right nostril. The left disposable nostril insert 38 and the right disposable nostril insert 40 can be detached from the proximal plug surface 34 and can be thrown away after at least one use. The left disposable nostril insert 38 and the right disposable nostril insert 40 are made of a transparent material, and the at least one nasal photo-biomodulator 36 is in optical communication with the left disposable nostril insert 38 and the right disposable nostril insert 40, which allows therapeutic electromagnetic radiation to be emitted from the at least one nasal photo-biomodulator 36, through the left disposable nostril insert 38 and the right disposable nostril insert 40, and into a user's nose.

As can be seen in FIGS. 1 through 4, the present invention may further comprise a length-adjustment mechanism 42, which is used to improve the fit of the headband 2 around a user's head. The length-adjustment mechanism 42 is operatively integrated into the band body 4, wherein the length-adjustment mechanism 42 is used to adjust a circumferential length of the band body 4.

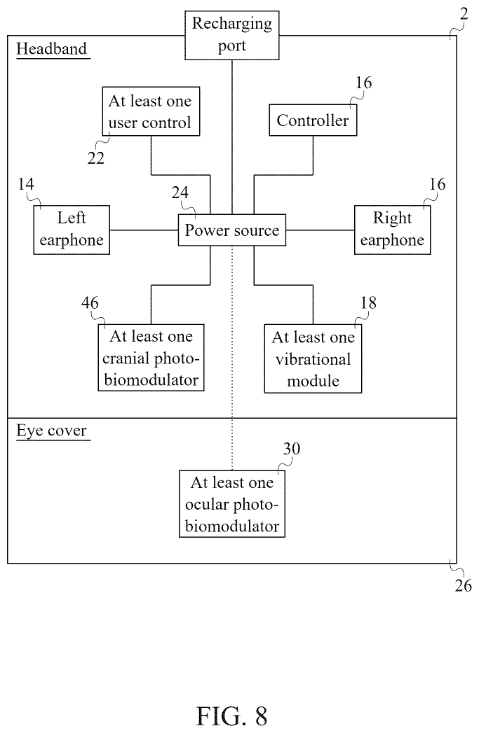

In some embodiments of the present invention, the power source 24 is further configured to better utilize electrical power resources available to the present invention. As can be seen in FIGS. 6 and 8, the power source 24 can be a portable power source (e.g., a battery) that is housed within the band body 4. The portable power source is preferably a rechargeable battery so that the present invention may further comprise a recharging port 44. The recharging port 44 is used to recharge the portable power source with an external electrical power source (e.g., an electrical outlet). The recharging port 44 is integrated into the band body 4 and is electrically connected to the power source 24 so that the recharging port 44 can be easily accessed by a user. The recharging port 44 is preferably a USB-C port, a USB port, or any other standardized charging port. The power source 24 can alternatively be a power cord that is tethered to the band body 4 in order to continuously receive electrical power from an electrical outlet.

As can be seen in FIGS. 6 through 8, the present invention may further comprise at least one cranial photo-biomodulator 46, which is used to stimulate a user's cranium in such a way to promote tissue repair, to reduce inflammation, to induce analgesia, to activate other therapeutic benefits, or a combination thereof. The at least one cranial photo-biomodulator 46 is mounted onto the inner band surface 8 so that the at least one cranial photo-biomodulator 46 is able to directly emit therapeutic electromagnetic radiation into a user's cranium. The at least one cranial photo-biomodulator 46 can be communicably coupled to the controller 20 so that the controller 20 is able to manage and/or modify therapeutic electromagnetic radiation being emitted into a user's cranium by the at least one cranial photo-biomodulator 46. The at least one cranial photo-biomodulator 46 is preferably configured to emit electromagnetic radiation with a 660-nanometer (nm) wavelength.

The present invention may further comprise a quantity of amethyst-crystal granules, which provide additional therapeutic benefits to a user of the present invention. The quantity of amethyst-crystal granules is preferably a quantity of crushed amethyst crystal. The quantity of amethyst-crystal granules is housed within the headband 2.

### Supplemental Description

It should be understood that the words “a”, “an”, and “the” also refer to the plural of the component in question, unless otherwise explicitly stated. For example, reference to “an earphone” should include reference to “one earphone”, “at least one earphone”, and “a plurality of earphones”. Unless otherwise explicitly indicated, it should be understood that any electronic component listed is electrically connected to a power source described herein to allow operation.

The neurological brain entrainment device may comprise a headband, an adjustment module, an earphone, an eye band, a vibrational band, a photo biomodulator, an intranasal device, a plurality of buttons, and a charging port.

The headband may comprise any cloth or fabric material that is well-known in the art. The headband may have an interior portion capable of housing electronics. In the ideal embodiment, the interior portion may be adapted to house a motherboard, the earphone, the vibrational band, and the photo biomodulator. The back of the headband may comprise the adjustment module, which may allow the size of the headband to be adjusted to fit various sizes of heads. The adjustment module may comprise any adjustment mechanism that is well-known in the art. The headband is adapted to be positioned over the eyes and forehead of the user and wrap around the back of the head.

The earphone may be positioned within the interior portion of the headband. The earphone may be adapted to emit sound frequencies, ideally being adapted to emit sound at 40 Hz. In the ideal embodiment, the earphone may be bio-formed, being molded into the shape of the interior of a human ear. This shape allows the earphone to more comfortably fit in the ear of a user. In the ideal embodiment, two earphones are used, one being positioned over each ear of the user.

The vibrational band may be positioned in the interior portion of the headband, on the sides of the head of the user. The vibrational band may produce low intensity vibrations that correlate to the frequency of the sound produced by the earphone.

The eye band may be attached to or detachably attached to the headband. The eye band may be positioned on the front of the headband, such that the eye band rests over the eyes of the user when the invention is in a position of use.

In the ideal embodiment, the eye band may further comprise the photo biomodulator. The photo biomodulator may produce photonic light at a gamma ray of 40 hz. In some embodiments, the frequency of the light may be adapted to match the frequency of the earphone and the frequency of the vibrational band.

The intranasal device may be either a separate piece that is detachably attachable to the headband or may be integrated into the headband or eye band. The intranasal device is adapted to be positioned within the nostril of the user. The intranasal device may further comprise a photo biomodulator similar to the photo biomodulator of the eye band. The photo biomodulator of the intranasal device may be adapted to produce photonic light at a gamma ray of 40 hz. In some embodiments, the frequency of the light may be adapted to match the frequency of the earphone and the frequency of the vibrational band. A plurality of disposable nodes may be used in the intranasal device to house the photo biomodulator. These nodes are ideally detachably attachable to the intranasal device to allow removal after use, facilitating hygiene of the device.

The charging port may be located in the rear of the device and may be adapted to allow connection to any charging adapter well-known in the art, such as USB-C, USB, or any other standard charging adapter. The charging port is ideally electrically connected to a battery located in or on the headband. The battery may be electrically connected to a motherboard located in the interior portion of the headband.

The motherboard may be adapted to receive and distribute any power, commands, or instructions to the various electronics and components described herein. For example, the motherboard may instruct and coordinate the earphones, photo biomodulator, and vibration pads.

The plurality of buttons may be positioned in any location on the Neurological Brain Entrainment Device. In the ideal embodiment, the plurality of buttons is electrically connected to the motherboard, being adapted to control the various functions of the brain entrainment device. An exemplary layout and configuration of the buttons is described as follows: a first button may be adapted to power on and off the device. A second button may be adapted to start and stop the photo biomodulator. A third button may be adapted to start the device for a selected period of time and cease function of the device once time has expired. A fourth button may be adapted to start and stop the parallel vibration of the vibration bands. A fifth button may be adapted to start a sleep mode, allowing the device to be adapted for use when a user sleeps, such as by turning on only vibration or sound functions, while leaving light functions off, or vice versa. A sixth button may be adapted to control the earphones. Though the ideal configuration of the plurality of buttons is described above, it should be understood that other configurations, numbers, and functions for the buttons are within the spirit and scope of the present invention.

The use of different frequency tones or a combination of differing frequency tones is within the spirit and scope of the present invention.

Although the invention has been explained in relation to its preferred embodiment, it is to be understood that many other possible modifications and variations can be made without departing from the spirit and scope of the invention as hereinafter claimed.

What is claimed is:

1. A neurological brain entrainment device comprising:
a headband;
a left earphone;
a right earphone
at least one vibrational module;
a controller;
at least one user control;
a power source;
the headband comprising a band body, an outer band surface, an inner band surface, an upper band rim, and a lower band rim;
the left earphone and the right earphone being integrated through the lower band rim and into the band body;
the left earphone and the right earphone being positioned opposite to each other about the band body;
the at least one vibrational module being integrating about the band body;
the at least one vibrational module being positioned adjacent to the left earphone and the right earphone;
the at least one user control being externally mounted to the outer band surface;
the controller being housed within the band body;
the power source being integrated into the band body;
the controller being electronically connected to the left earphone, the right earphone, the at least one vibrational module, and the at least one user control; and
the power source being electrically connected to the controller, the left earphone, the right earphone, the at least one vibrational module, and the at least one user control.

2. The neurological brain entrainment device as claimed in claim 1, wherein the left earphone and the right earphone are configured to emit a 40-Hertz (Hz) sound.

3. The neurological brain entrainment device as claimed in claim 1, wherein the left earphone is ergonomically shaped to securely fit into a user's left ear, and wherein the right earphone is ergonomically shaped to securely fit into a user's right ear.

4. The neurological brain entrainment device as claimed in claim 1, wherein the at least one vibrational module is configured to vibrate at 40 Hz.

5. The neurological brain entrainment device as claimed in claim 1 further comprising:
the at least one vibrational module being a left vibrational module and a right vibration module; and
the left vibrational module and the right vibrational module being positioned offset from each other about the band body.

6. The neurological brain entrainment device as claimed in claim 1 further comprising:
an eye cover;
the eye cover being mounted adjacent to the lower band rim; and
the eye cover being positioned in between the left earphone and the right earphone about the band body.

7. The neurological brain entrainment device as claimed in claim 6, wherein the eye cover is hingedly connected to the lower band rim.

8. The neurological brain entrainment device as claimed in claim 6 further comprising:
at least one ocular photo-biomodulator;
the eye cover comprising a proximal cover surface; and
the at least one ocular photo-biomodulator being mounted across the proximal cover surface.

9. The neurological brain entrainment device as claimed in claim 8, wherein the at least one ocular photo-biomodulator is communicably coupled to the controller.

10. The neurological brain entrainment device as claimed in claim 8, wherein the at least one ocular photo-biomodulator is configured to emit electromagnetic radiation with a 660-nanometer (nm) wavelength.

11. The neurological brain entrainment device as claimed in claim 1 further comprising:
an intranasal plug; and
the intranasal plug being positioned offset from the band body.

12. The neurological brain entrainment device as claimed in claim 11, wherein the intranasal plug is tethered to the band body.

13. The neurological brain entrainment device as claimed in claim 11 further comprising:
at least one nasal photo-biomodulator;
the intranasal plug comprising a proximal plug surface; and
the at least one nasal photo-biomodulator being mounted across a proximal plug surface.

14. The neurological brain entrainment device as claimed in claim 13 further comprising:
a left disposable nostril insert;
a right disposable nostril insert;
the left disposable nostril insert and the right disposable nostril insert being made of a transparent material;
the left disposable nostril insert and the right disposable nostril insert being attached onto the proximal plug surface; and
the at least one nasal photo-biomodulator being in optical communication with the left disposable nostril insert and the right disposable nostril insert.

15. The neurological brain entrainment device as claimed in claim 13, wherein the at least one nasal photo-biomodulator is communicably coupled to the controller.

16. The neurological brain entrainment device as claimed in claim 13, wherein the at least one nasal photo-biomodulator is configured to emit electromagnetic radiation with a 660-nm wavelength.

17. The neurological brain entrainment device as claimed in claim 1 further comprising:
a length-adjustment mechanism; and
the length-adjustment mechanism being operatively integrated into the band body, wherein the length-adjustment mechanism is used to adjust a circumferential length of the band body.

18. The neurological brain entrainment device as claimed in claim 1 further comprising:
the power source being a portable power source; and
the power source being housed within the band body.

19. The neurological brain entrainment device as claimed in claim 18 further comprising:
a recharging port;
the recharging port being integrated into the band body; and
the recharging port being electrically connected to the power source.

20. The neurological brain entrainment device as claimed in claim 1 further comprising:
the power source being a power cord; and
the power source being tethered to the band body.

21. The neurological brain entrainment device as claimed in claim 1 further comprising:
at least one cranial photo-biomodulator; and
the at least one cranial photo-biomodulator being mounted onto the inner band surface.

22. The neurological brain entrainment device as claimed in claim 21, wherein the at least one ocular photo-biomodulator is communicably coupled to the controller.

23. The neurological brain entrainment device as claimed in claim 21, wherein the at least one ocular photo-biomodulator is configured to emit electromagnetic radiation with a 660-nanometer (nm) wavelength.
